(19) United States
(12) Patent Application Publication
(10) Pub. No.: US 2016/0089172 A1
(43) Pub. Date: Mar. 31, 2016

(54) DEVICES AND METHODS FOR APPLYING SUCTION

(71) Applicant: Boston Scientific Scimed, Inc., Maple Grove, MN (US)

(72) Inventor: James E. WINDHEUSER, Hopkinton, MA (US)

(73) Assignee: Boston Scientific Scimed, Inc., Maple Grove, MN (US)

(21) Appl. No.: 14/869,425

(22) Filed: Sep. 29, 2015

Related U.S. Application Data
(60) Provisional application No. 62/057,767, filed on Sep. 30, 2014.

(51) Int. Cl. A61B 17/221

(52) U.S. Cl. CPC A61B 17/221; A61B 2017/22079

(57) ABSTRACT

A medical retrieval device may include a member extending longitudinally between a proximal end and a distal end. The member may include at least one lumen configured to be fluidly coupled to a vacuum source and to receive a medical tool therethrough. The member may be configured to transition between a compressed configuration and an expanded configuration, wherein in the expanded configuration, the distal end may have a larger cross-sectional area than the proximal end.

### CROSS-REFERENCE TO RELATED APPLICATIONS

The application claims the benefits of priority from U.S. Provisional Application No. 62/057,767, filed on Sep. 30, 2014, the entirety of which is incorporated herein by reference.

### TECHNICAL FIELD

The disclosure generally relates to medical retrieval devices for retrieving material in a body of a patient. More particularly, the disclosure generally relates to medical retrieval devices including a source of suction for manipulating, moving, and/or capturing material within a body of a patient, and related methods of use.

### BACKGROUND

Medical retrieval devices are often utilized for removing organic material (e.g., blood clots, tissue, and biological concretions such as urinary, biliary, and pancreatic stones) and inorganic material (e.g., components of a medical device or other foreign matter), which may obstruct or otherwise be present within a patient's body cavities. For example, concretions can develop in certain parts of the body, such as in the kidneys, pancreas, ureter, and gallbladder. Minimally invasive medical procedures are used to remove these concretions through natural orifices, or through an incision, such as during a percutaneous nephrolithotomy (PNCL) procedure. Other procedures may include endoscopic retrograde cholangiopancreatography (ERCP) which is a procedure for treating the bile and pancreatic ducts of a patient. Further, lithotripsy and ureteroscopy, for example, are used to treat urinary calculi (e.g., kidney stones) in the ureter of a patient.

Conventional medical retrieval devices may intend to retrieve the stone or other material that is positioned within difficult to access anatomy of a patient or is lodged or embedded within tissue of the patient, but may be unable to obtain the requisite access. The medical retrieval device also may not be sufficiently flexible to traverse patient anatomy, etc. In such cases, medical professionals may be forced to retract the medical retrieval device and attempt to use an additional or alternative medical retrieval device, and/or access the patient anatomy through more invasive procedures such as, for example, open surgery. Retracting the medical retrieval device may add unnecessary time to a procedure, while more invasive procedures may be riskier and may result in increased danger and infection for a patient.

### SUMMARY

Examples of the present disclosure relate to, among other things, medical devices for diagnosing and/or treating internal areas of a subject's body. The medical devices may include suction capability. Each of the examples disclosed herein may include one or more of the features described in connection with any of the other disclosed examples.

In one example, a medical retrieval device may include a member extending longitudinally between a proximal end and a distal end. The member may include at least one lumen configured to be fluidly coupled to a vacuum source and to receive a medical tool therethrough. The member may be configured to transition between a compressed configuration and an expanded configuration, wherein in the expanded configuration, the distal end may have a larger cross-sectional area than the proximal end, and wherein the member may be trumpet shaped.

Examples of the medical retrieval device may include one or more of the following features: the member may include a braided material; the member may include a transition portion between the proximal end and the distal end, wherein the transition portion may define a sloping or arcuate portion having a degree of sloping or curvature between 45° and 60°; the member may include one or more of plastic, rubber, and polyurethane, Pebax, PTFE, stainless steel, and a metal alloy; the at least one lumen may be a first lumen and may extend along a central longitudinal axis of the member; the at least one lumen may include at least one second lumen, the at least one second lumen may be positioned along an inner circumference of the member, and the at least one second lumen may be configured to receive the medical tool therethrough; the first lumen may have a first cross-sectional shape and the at least one second lumen may have a second cross-sectional shape, wherein the first cross-sectional shape may be different than the second cross-sectional shape; the member may include a circular cross-sectional shape; an expandable member may be coupled to the distal end of the member; a vacuum source may be coupled to the member, wherein the vacuum source may include a regulator, and wherein manipulation of the regulator may vary suction provided by the vacuum source through the at least one lumen; the vacuum source may be coupled to the member through one or more of an air tight compression fitting, a gasket, and a luer lock; the member may include at least two second lumens; the at least two second lumens may be equidistantly spaced along an inner circumference of the member; the member may include at least three second lumens; and the member may include radiopaque material.

In another example, a medical retrieval device may include an elongate member extending longitudinally between a proximal end and a distal end. The member may include a plurality of longitudinally-extending lumens, and may be configured to transition between a compressed configuration and an expanded configuration. In the expanded configuration, the distal end may have a larger cross-sectional area than the proximal end. A mesh may extend along at least a distal portion of the member and at least one lumen of the plurality of lumens may be configured to be in fluid communication with a vacuum source.

Examples of the medical retrieval device may include one or more of the following features: the mesh may be coupled to at least one of an inner surface of the member and an outer surface of the member; the mesh may be embedded within the member; the member may be trumpet shaped; the at least one lumen may extend along a central longitudinal axis of the member; and the plurality of lumens may further include at least one additional lumen positioned along an inner circumference of the member.

In another example, a method may include delivering a member to an area within a patient. The member may extend longitudinally between a proximal end and a distal end and include a first lumen and at least one second lumen. The method may also include transitioning the member from a compressed configuration to an expanded configuration, wherein in the expanded configuration, the distal end has a larger cross-sectional area than the proximal end. The method may further include applying vacuum pressure through the first lumen to suction an object at least partially into the distal end, and deploying a medical tool through the at least one second lumen.

Examples of the method may include one or more of the following features: manipulating the object via the medical tool; repositioning the member to a second area, and ceasing vacuum pressure through the first lumen and releasing the object at the second area; manipulating the object via the medical tool at the second area; and at least a portion of the member may be braided, and removing the object from the area of interest within the patient.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is an illustration of an exemplary medical system including a medical tool extending therein, according to the present disclosure;

FIG. 2 is a side-view of the exemplary medical tool of FIG. 1; and

FIG. 3 is a cross-sectional view of the exemplary medical tool of FIG. 2, taken along line A-A.

### DETAILED DESCRIPTION

Examples of the present disclosure relate to medical devices for diagnosing and/or treating internal areas of a subject's body. The medical devices may include suction capability.

Reference will now be made in detail to examples of the present disclosure described above and illustrated in the accompanying drawings.

The terms “proximal” and “distal” are used herein to refer to the relative positions of the components of an exemplary medical device. When used herein, “proximal” refers to a position relatively closer to the exterior of the body or closer to a user using the medical device. In contrast, “distal” refers to a position relatively further away from the user using the medical device, or closer to the interior of the body. As used in this disclosure, the term “about” may encompass a range of values between plus or minus 5% of a defined value.

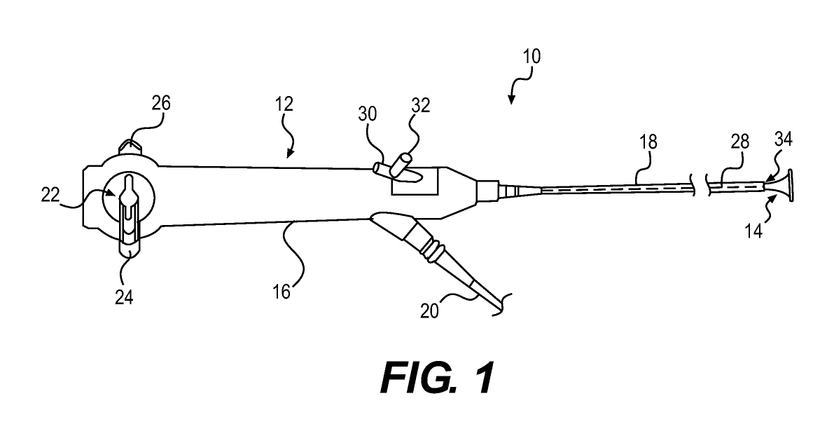

FIG. 1 shows an exemplary medical system 10. Medical system 10 may include a medical device 12 configured to allow a medical professional to perform medical diagnoses and/or treatments on a subject. For example, medical device 12 may include any device configured to allow a user to access and view internal areas of a subject's body. Additionally or alternatively, medical device 12 may include any device configured to deliver medical instruments, such as, for example, suction tool 14, biopsy forceps, graspers, baskets, snares, probes, scissors, retrieval devices, lasers, and/or other tools, into a subject's body. Medical device 12 may be inserted into a variety of body openings, lumens, and/or cavities. For example, medical device 12 may be inserted into any portion of a urinary tract, such as a ureter, a gastrointestinal lumen, such as an esophagus, a vascular lumen, and/or an airway.

According to aspects of the present disclosure, medical device 12 may be a ureteroscope. In some contemplated examples, medical device 12 may be a sterile, single-use, and disposable ureteroscope. Alternatively, medical device 12 may be a multiple-use, non-disposable ureteroscope. Other types of devices, however, may be substituted for the ureteroscope, including, as examples, an endoscope, a hysteroscope, a uteroscope, a bronchoscope, a cystoscope, and similar devices. Such devices may be single-use and disposable, or multiple-use and non-disposable.

Medical device 12 may include a handle assembly 16 operably connected to an elongate tubular member 18. Tubular member 18 may include, for example, a catheter, and may be configured to be at least partially inserted into a subject's body and navigated to an internal area therein. Tubular member 18 may be flexible and may be maneuvered into, through, and out of the subject's body. Tubular member 18 may be configured, for example, to traverse tortuous anatomical lumens of the subject's body. Tubular member 18 may be uniformly flexible, or may include a plurality of portions having varying degrees of flexibility or rigidity. Tubular member 18 may have a substantially uniform cross-sectional configuration along its length, but it may taper distally. In other examples, the cross-sectional shape may vary along the length of tubular member 18. For example, in some examples, a proximal portion of tubular member 18 may have an ovular cross-section shape while a distal portion of tubular member 18 may have a circular cross-sectional shape. Other sizes, shapes, and arrangements are contemplated and within the scope of this disclosure.

Medical device 12 may also include an imaging assembly including an image sensor (not shown) at a distal end of tubular member 18. An image sensor may be at least partially mounted within, or embedded within, the distal end of tubular member 18. It is also contemplated that tubular member 18 may have a distal end cap (not shown), and the image sensor may be positioned therein. The image sensor may view an area distal to the distal end of tubular member 18. The image sensor may be any suitable type of image sensor configured to capture images and/or full-motion video images in digital or any other suitable format such as, for example, a charged couple device (“CCD”) or a complementary metal oxide semiconductor (“CMOS”) image sensor. The image sensor may be configured to send and/or receive data via appropriate conductors and/or deliver such information to an external component, such as a user interface and/or display screen, via a connector 20.

Further, the medical device 12 may include an illumination assembly including an illumination unit, such as, for example, a light-emitting diode (“LED”) (not shown) and an illumination fiber (not shown). The illumination fiber may extend longitudinally within tubular member 18 and deliver light emitted by the LED towards a distal end of tubular member 18 where the light may be emitted from the distal tip of the illumination fiber to areas around the distal end of tubular member 18. The illumination fiber may include an optical fiber made of plastic, glass, or any other suitable light transmissive material.

Handle assembly 16 may also include a steering mechanism 22. Steering mechanism 22 may be configured to control the steering and deflection of tubular member 18. Steering mechanism 22 may include a first actuator 24 and a second actuator 26 configured to control deflection of a distal portion of tubular member 18 between a substantially linear configuration and a variety of curved, angled, or bent configurations, in a variety of different directions relative to a longitudinal axis 28 of tubular member 18 via control wires, Bowden cables, and/or push-pull members (not shown). For example, actuating first actuator 24 in opposing directions may cause the distal portion of tubular member 18 to deflect in opposing directions along a first plane. Actuating second actuator 26 in opposing directions may cause the distal portion of tubular member 18 to deflect in opposing directions along a second plane different than the first plane. Accordingly, steering mechanism 22 may provide four-way steering of the distal portion of tubular member 18. The ability to steer allows the user to achieve visualization of almost any internal area in the subject's body.

Handle assembly 16 may also include ports 30 and 32. Ports 30 and 32 may provide access to one or more channels, such as working channel 34 extending through tubular member 18. For example, port 30 and/or port 32 may provide access for one or more medical instruments or tools, such as suction tool 14, into one or more channels, including working channel 34 extending through tubular member 18 and out the distal tip of tubular member 18 as shown in FIG. 1. Additionally or alternatively, port 30 and/or port 32 may provide access into one or more working channels, such as working channel 34, for delivering a suitable fluid, such as a liquid or gas, for irrigation and insufflation purposes, respectively, to and out of the distal tip of tubular member 18. It is also contemplated that port 30 and/or port 32 may be in fluid communication with one or more working channels, such as working channel 34, for withdrawing material from tubular member 18 and/or an area near the distal tip of tubular member 18, using suction. Tubular member 18 may include one or more additional channels for receiving other components.

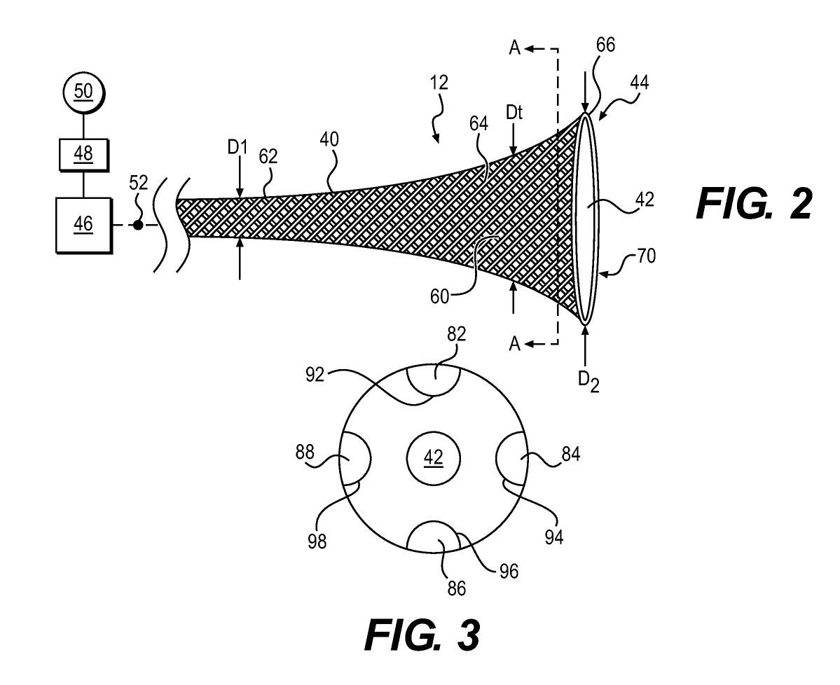

As best shown in FIG. 2, suction tool 14 may include a longitudinally extending hollow member 40 defining a channel 42 extending therethrough. Channel 42 may extend from a proximal end (not shown) of hollow member 40 to a distal end 44 of hollow member 40. Channel 42 may be configured to convey suction fluid therethrough as will be described in further detail below.

Hollow member 40 may be configured to be delivered to a location within the body of the patient via the working channel 34 of the medical device 10. Accordingly, hollow member 40 may be flexible and configured to bend along with tubular member 18. Accordingly, hollow member 40 may include any appropriate material such as, for example, biocompatible plastic, biocompatible rubber, metal, braided catheter material, composites, and biocompatible polyurethane. In some examples, hollow member 40 may include one or more of Pebax, PTFE, or Polyurethane.

A proximal end (not shown) of the hollow member 40 may be coupled to a vacuum source 46. Vacuum source 46 may provide suction through the channel 42. Vacuum source 46 may be fluidly coupled to hollow member 40 and channel 42 through any appropriate means including, for example, an air tight compression fitting, gasket, and/or a luer lock. Vacuum source 46 may include a regulator 48 which may allow a medical professional to adjust and/or vary the suction provided by the vacuum source 46 through channel 42. For example, regulator 48 may include a suction adjustment dial 50. In use, a medical professional may rotate or otherwise actuate suction adjustment dial 50 to adjust the suction pressure or flow rate of vacuum source 46. In addition, a control valve 52 may be positioned between vacuum source 46 and hollow member 40 such that a medical professional may selectively prevent and allow vacuum pressure to be applied to hollow member 40 when desired. Control valve 52 may include any appropriate valve such as, a pinch valve or stop valve. It will be understood that additional or alternative control valves 52 may be used and/or located at additional and/or alternative positions. Further, the vacuum source 46 may be located near the patient or alternatively, the vacuum source 46 can be located remotely. In some arrangements, vacuum source 46 may include existing suction capabilities of medical device 10.

Channel 42 may be configured to resist collapse when suction is provided through channel 42 by vacuum source 46. For example, hollow member 40 may be reinforced to resist collapse of channel 42 when suction is provided through channel 42. Accordingly, hollow member 40 may include a braid and/or mesh 60 or other construction configured to circumferentially and longitudinally support channel 42 and prevent collapse when suction is applied via vacuum source 46. Mesh 60 may be made of stainless steel, biocompatible plastic, biocompatible rubber, biocompatible polyurethane, and/or other material(s). The mesh 60 can be, for example, formed by two or more cords that are woven together to form the mesh 60 with each cord having one or more, for example three or more, component strands. Each of the strands could be thin wire, metal, metal alloy, biocompatible plastic, biocompatible rubber, and biocompatible polyurethane, for example. The mesh 60 may reinforce the hollow member 40 so that the hollow member 40 does not collapse when suction is provided through channel 42 by the vacuum source 46. The cords of the mesh 60 can be either spaced closely together to form a dense mesh 60 or spaced far apart to form a sparse or open mesh 60. The mesh 60 can be affixed to an inner surface, an outer surface, or embedded within hollow member 40. Alternatively, the mesh 60 may in itself form the hollow member 40 and may be referred to as a braided or mesh hollow member. For example, while in some cases, a separate hollow member 40 may be reinforced with the mesh 60, in other cases no separate hollow member 40 is required and mesh 60 may itself be configured to be coupled to a vacuum source 46. In such cases, mesh 60 may form a dense mesh such that vacuum pressure may be maintained within mesh 60. It is understood, therefore, that any description herein of hollow member 40 may apply with equal force to mesh 60. Additionally, the mesh 60 may be configured to aid hollow member 40 to transition between a first compressed configuration and a second expanded configuration, as will be described in further detail below.

Hollow member 40 may be configured to transition between a first compressed configuration (not shown) and a second expanded configuration (FIGS. 1 and 2). In the compressed configuration, an outer radial dimension (e.g., diameter) of hollow member 40 may be a constrained to a first value. In the expanded configuration, however, the outer radial dimension (e.g., diameter) of hollow member may expand to a second value, larger than the first value. In other words, when positioned within the working channel 34 and/or an additional sheath (not shown), the hollow member 40 may be retained in the compressed configuration and prevented from expanding to the expanded configuration. Once extended distally of the working channel 34 and/or the additional sheath, radial compression of hollow member 40 is relieved and hollow member 40 may freely expand to the expanded configuration. Additionally, the braid and/or mesh 60 may aid hollow member 40 to transition between the compressed configuration and the expanded configuration. For example, the mesh 60, due to its material properties, may assist hollow member 40 to expand or open when deployed distally of the working channel 34 and/or additional sheath.

As shown most clearly in FIG. 2, distal end 44 of hollow member 40 may include a trumpet-shaped configuration. For example, as shown in FIG. 2, in the expanded configuration, the hollow member may have a proximal portion 62, a transition portion or medial portion 64 and a distal portion 66. As shown, the proximal portion 62 may include a first diameter D₁, and the distal portion 66 may include a second diameter D₂, while the transition portion 64 may have a transition diameter Dₜ. The second diameter D₂ may be larger than the first diameter D₁. Additionally, the transition diameter Dₜ may have a dimension or size between the first diameter D₁ and the second diameter D₂. Accordingly, a distal end 44 of the hollow member may have a trumpet-shaped configuration. In other words, a cross-sectional area of the distal end 44 may be larger than a cross-sectional area of a proximal end of the hollow member 40 in the expanded configuration.

The transition portion 64 may connect the proximal portion 62 and the distal portion 64 and define a sloping or arcuate portion of hollow member 40. The degree of slope or curvature of transition portion 64 may be any appropriate degree. For example, the degree of sloping or curvature may be between a range having a low end approaching about 0° and a high end approaching about 90°. Accordingly, the degree of sloping or curvature may be between about 5° and about 85°. As shown in FIG. 2, transition portion 64 may smoothly and gradually transition from first diameter D₁ to second diameter D₂. Accordingly, the degree of sloping or curvature may be between about 45° and about 60°. In other examples, transition portion 64 may sharply and/or abruptly transition from first diameter D₁ to second diameter D₂. In such arrangements, for example, the degree of sloping or curvature may be greater than about 60°.

Additionally, distal end 44 of hollow member 40 may include an expandable member 70. Expandable member 70 may include a ring or other shaped member positioned adjacent distal end 44 of hollow member 40. For example, in some arrangements, expandable member may be embedded or otherwise retained within distal end 44 of hollow member 40. In other arrangements, expandable member 70 may be attached, coupled, or otherwise affixed to an interior surface or an exterior surface of distal end 44 of hollow member 40. Expandable member 70 may be included so as to assist hollow member 40 to transition between the compressed configuration to the expanded configuration, and to prevent hollow member 40 from collapsing when suction is applied to channel 42 via vacuum source 46. Accordingly, expandable member 70 may include any appropriate readily expanded material. For example, in some configurations, expandable member 70 may include a shape-memory material such as, for example, Nitinol, which may be formed into a first shape (e.g., a compressed or collapsed shape) and upon experiencing a change in temperature, may transition to a transition shape (e.g., an expanded shape). Other materials may also be used without departing from the scope of this disclosure.

In addition, one or more portions of hollow member 40 may be coated with or made of radiopaque material. For example, expandable member 70 and/or distal end 44 of hollow member 40 may include radiopaque material which may facilitate locating hollow member 40 with a fluoroscope when the hollow member 40 is positioned within a patient. The radiopaque material can be painted on, wrapped around, and/or embedded in hollow member 40. In some examples, the radiopaque material can be tungsten filled ink, bismuth subcarbonate, and/or barium sulfate. In other examples, the radiopaque material can be a platinum and/or tantalum metal band.

As shown in FIG. 3, the hollow member 40 may define a plurality of lumens 82, 84, 86, and 88, as well as channel 42. While four lumens 82-88 have been depicted, it is understood that more or fewer lumens may be defined by hollow member 40 in accordance with this disclosure. For example, any appropriate number of lumens may be used such as between one and ten lumens. Lumens 82-88 may each be configured for passing one or more medical tools therethrough. For example, one or more of lumens 82-88 may be configured to pass biopsy forceps, graspers, baskets, snares, probes, scissors, retrieval devices, lasers, and/or other tools into a subject's body. Accordingly, each lumen 82-88 may be sized and/or shaped to readily pass one or more such tools therethrough. Additionally or alternatively, any one or more of lumens 82-88 may be configured to deliver one or more of saline, water, insufflation gas, medicament, and cleaning solution into a subject's body. In these arrangements, such lumens may be coupled to an external source of saline, water, insufflation gas, medicament, and cleaning solution. The source(s) may or may not be pressurized. It should be understood, however, that lumens 82-88 may be omitted, such that channel 42 is the only lumen through hollow member 40. In such an embodiment, channel 42 may provide suction, deliver one or more materials, pass one or more tools/instruments, and/or perform any other function that may otherwise be performed by one or more additional lumens like lumens 82-88.

As shown in FIG. 3, each of lumens 82-86 may be circumferentially spaced about the inner circumference of hollow member 40 and may extend from a proximal end to distal end 44 of hollow member 40. While lumens 82-88 are depicted as equidistantly spaced about the inner circumference of hollow member 40, it is understood that lumens 82-88 may be positioned anywhere along the inner circumference of hollow member 40. For example, lumens 82-88 may be unequally spaced about the inner circumference of hollow member 40. Additionally or alternatively, each lumen 82-88 may be similarly sized and shaped. For example, as shown in FIG. 3, each lumen 82-88 may be semi-circular shaped and about the same size. In other arrangements, however, one or more lumens 82-88 may be differently shaped or sized. For example, any one or more of lumens 82-88 may be circular or elliptically shaped. Additionally, the shape of lumens 82-88 may be varied. For example, one or more lumens 82-88 may be semi-circular shaped while others may have a circular or elliptical shape. Additionally, any one or more of lumens 82-88 may include a lubricious coating or material to facilitate insertion and/or removal of tools therethrough. Further, as shown in FIG. 3, channel 42 may be centrally located within hollow member 40. For example, channel 42 may be positioned along a central axis of hollow member 40. In other examples, however, channel 42 may be positioned anywhere within hollow member 40. For example, channel 42 may be offset from a central axis of hollow member 40.

Additionally, lumens 82-88 may be formed monolithically within hollow member 40. For example, lumens 82-88 may be extruded as a one-piece construction with hollow member 40 and/or may be braided as a one-piece braid and/or mesh with mesh 60. In other words, hollow member 40 and lumens 82-88 may be a unitary construction. Alternatively, lumens 82-88 may be separately formed and coupled to hollow member 40. For example, each of lumens 82-88 may be individually extruded and/or braided and then attached to hollow member 40 through any appropriate coupling mechanism, such as, for example, mechanical fasteners and/or adhesives. Additionally, each lumen 82-88 may define a lumen wall 92-98, respectively. For example, construction of each lumen 82-88 may result in the formation of a lumen wall 92-98 thereby defining each lumen 82-88.

In use, a medical professional may insert a distal end of tubular member 18 into the body of a patient. For example, the medical professional may deliver the distal end of tubular member 18 into a urethra of a patient. If so desired, the medical professional may then advance tubular member 18 increasingly distally into the patient such that a distal end of tubular member may be positioned at an appropriate location within the body of a patient. For example, the medical professional may advance tubular member 18 such that it is passed into and through the urinary bladder and/or, into and through the ureter and/or kidney of the patient. To facilitate positioning of the tubular member 18 within the body of the patient, the medical professional may use first actuator 24 and second actuator 26 of steering mechanism 22 to control deflection of the distal end of tubular member 18 as described above.

Once positioned at a desired location within the body of the patient, the medical professional may identify one or more of blood clots, tissue, and biological concretions such as stones and/or inorganic materials positioned within the body of the patient through the use of an imaging assembly as described above. If such an object of interest is identified, the medical professional may insert hollow member 40, while in the compressed configuration, into and through port 30 or port 32 and working channel 34. In some arrangements, hollow member 40 may be encased or surrounded by an additional sheath (not shown) so as to facilitate insertion into and distal advancement through working channel 34 of tubular member 18. A proximal end of hollow member 40, extending through port 30 or 32, may then be coupled to vacuum source 46 as noted above. In some examples, however, hollow member 40 may be fluidly coupled to vacuum source 46 prior to insertion into and through working channel 34.

Next, a medical professional may advance hollow member 40 distally of working channel 34. In examples in which an additional sheath is used, a medical professional may retract the additional sheath relative to the hollow member as noted above. Once the hollow member 40 extends distally of working channel 34 and/or the additional sheath, hollow member 40 may transition between the compressed configuration and the expanded configuration (FIGS. 1 and 2). For example, once extended distally of the working channel 34 and/or the additional sheath, radial compression of hollow member 40 is relieved and hollow member 40 may freely expand to the expanded configuration in which the distal end 44 includes a trumpeted shape as shown in FIG. 2. Additionally, as noted above, braid and/or mesh 60 may aid hollow member 40 to transition between the compressed configuration and the expanded configuration.

Once hollow member 40 transitions to the expanded configuration, the medical professional may open control valve 52 so as to allow vacuum pressure from vacuum source 46 to be applied through channel 42. As noted above, the amount of vacuum pressure applied through the channel 42 may be adjusted via regulator 48 and dial 50.

Next, the medical professional may use hollow member 40 to either remove, retain, or relocate an object of interest. For example, if the object of interest is sufficiently small so as to easily pass through channel 42 of hollow member 40, the medical professional may direct distal end 44 of hollow member 40 towards and sufficiently close to the object of interest so as to suck the object of interest into hollow member 40. In some examples, the object of interest may be small enough to be sucked proximally through the hollow member 40 and towards a proximal end of the hollow member 40 and removed therefrom. Once the object of interest has been removed form hollow member 40, the procedure may be repeated as needed.

Alternatively, the object of interest may be small enough to be received, in whole or in part, within the trumpeted distal end 44 of hollow member 40 but not small enough to be passed proximally through channel 42 of hollow member 40. In these examples, a medical professional may direct distal end 44 of hollow member 40 towards and sufficiently close to the object of interest so as to suck at least a portion of the object of interest into distal end 44 of hollow member 40. Once positioned therein, the object of interest and hollow member 40 may be proximally retracted through working channel 34 and removed from tubular member 18 as needed. Once removed from working channel 34, the object of interest may be removed from hollow member 40, and then hollow member 40 may be reinserted into working channel 34 to repeat the procedure as needed. To release the object of interest from hollow member 40, suction may be discontinued.

Additionally or alternatively, a medical professional may use hollow member 40 to relocate or reposition the object of interest within the body of the patient. For example, a medical professional may direct distal end 44 of hollow member 40 towards and sufficiently close to the object of interest so as to suck the object of interest, in whole or in part, into distal end 44 of hollow member 40. Once positioned therein, the medical professional may then reposition the distal end of tubular member 18 with hollow member 40 positioned therein and retaining the object of interest within distal end 44 and/or channel 42. The purpose of repositioning the distal end of tubular member 18 is to move the object of interest to an area in the patient's body where it may be more readily accessed, removed, and/or treated. For example, in situations in which the object of interest is located within tortuous or difficult to navigate anatomy of the patient, the medical professional may reposition the object of interest so as to allow a medical tool to easily reach and/or treat the object of interest. In such cases, once repositioned, one or more tools may be passed through any one or more of lumens 82-88 so as to reach the repositioned object of interest. For example, a basket (not shown) may be directed through lumen 82 so as to capture and or retain the object of interest. Once retained within the basket or other tool, the object of interest may be removed from the body of the patient in whole, or may be broken apart into smaller pieces via another tool, and the smaller pieces removed from the body of the patient. It is understood that in some cases, suction through channel 42 may be discontinued prior to capture, retention, an/or manipulation of the object of interest by the one or more tools.

The many features of the present disclosure are apparent from the detailed specification, and thus, it is intended by the appended claims to cover all such features and advantages of the present disclosure which fall within the true spirit and scope of the present disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

We claim:

1. A medical retrieval device, comprising:
a member extending longitudinally between a proximal end and a distal end, the member including at least one lumen configured to be fluidly coupled to a vacuum source and to receive a medical tool therethrough;
wherein the member is configured to transition between a compressed configuration and an expanded configuration, wherein in the expanded configuration, the distal end has a larger cross-sectional area than the proximal end, wherein the member is trumpet shaped.

2. The medical retrieval device of claim 1, wherein the member includes a braided material.

3. The medical retrieval device of claim 1, wherein the member includes a transition portion between the proximal end and the distal end, wherein the transition portion defines a sloping or arcuate portion having a degree of sloping or curvature between 45° and 60°.

4. The medical retrieval device of claim 1, wherein the member comprises one or more of plastic, rubber, and polyurethane, Pebax, PTFE, stainless steel, and a metal alloy.

5. The medical retrieval device of claim 1, wherein the at least one lumen includes a first lumen, the first lumen extends along a central longitudinal axis of the member, and the first lumen is configured to be fluidly coupled to the vacuum source.

6. The medical retrieval device of claim 5, wherein the at least one lumen includes at least one second lumen, the at least one second lumen is positioned along an inner circumference of the member, and the at least one second lumen is configured to receive the medical tool therethrough.

7. The medical retrieval device of claim 6, wherein the first lumen has a first cross-sectional shape and the at least one second lumen has a second cross-sectional shape, wherein the first cross-sectional shape is different than the second cross-sectional shape.

8. The medical retrieval device of claim 1, further comprising:
an expandable member coupled to the distal end of the member.

9. The medical retrieval device of claim 1, further including:
a vacuum source coupled to the member, wherein the vacuum source includes a regulator, and wherein manipulation of the regulator varies suction provided by the vacuum source through the at least one lumen.

10. A medical retrieval device, comprising:
an elongate member extending longitudinally between a proximal end and a distal end, wherein the member includes a plurality of longitudinally-extending lumens and is configured to transition between a compressed configuration and an expanded configuration, wherein in the expanded configuration, the distal end has a larger cross-sectional area than the proximal end; and
a mesh extending along at least a distal portion of the member;
wherein at least one lumen of the plurality of lumens is configured to be in fluid communication with a vacuum source.

11. The medical retrieval device of claim 10, wherein the mesh is coupled to at least one of an inner surface of the member and an outer surface of the member.

12. The medical retrieval device of claim 10, wherein the mesh is embedded within the member.

13. The medical retrieval device of claim 10, wherein the member is trumpet shaped.

14. The medical retrieval device of claim 10, wherein the at least one lumen extends along a central longitudinal axis of the member.

15. The medical retrieval device of claim 10, wherein the plurality of lumens comprises at least one additional lumen positioned along an inner circumference of the member.

16. A method, comprising:
delivering a member to an area of interest within a patient, the member extending longitudinally between a proximal end and a distal end and including a first lumen and at least one second lumen;
transitioning the member from a compressed configuration to an expanded configuration, wherein in the expanded configuration, the distal end has a larger cross-sectional area than the proximal end;
applying vacuum pressure through the first lumen to suction an object at least partially into the distal end; and
deploying a medical tool through the at least one second lumen.

17. The method of claim 16, further comprising:
manipulating the object via the medical tool.

18. The method of claim 16, further comprising:
repositioning the member to a second area; and
ceasing vacuum pressure through the first lumen and releasing the object at the second area.

19. The method of claim 18, further comprising:
manipulating the object via the medical tool at the second area.

20. The method of claim 16, wherein at least a portion of the member is braided, and further comprising:
removing the object from the area within the patient.
